SEQUENCE LISTING (4 sequences)

SEQ ID NO: 1 - DNA, 2478 bp, Homo sapiens
ctctgagtcgtctgagctcactgtgagcaaaatcccacagtggaaactcttaagcctctgcgaagtaaatcattcttgtgaatgtgacacacgatctctccagtttccatatgttgagattctacttattcatcagtttgttgtgcttgtcaagatcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacaggcctcgaccctgcaggccctttattcaacgggaaacctcaccaagacagattagatcccagtgatgcgcagtttgttgatgtcatccattccgacactgatgcactgggctacaaggagccattaggaaacatagacttctacccaaatggaggattggatcaacctggctgccccaaaacaatattgggaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggctattatgctgataattggaaagaccatctaagggggaaagatcctccaatgacgaaggcattctttgacacagctgaggagagcccattctgcatgtatcattactttgtggatattataacatggaacaagaatgtaagaagaggggacattaccatcaaattgagagacaaagctggaaacaccacagaatccaaaatcaatcatgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggcctcagctgtgtcggtatgatcttgtcctgatggaaaacgttgaaacagtcttccaacctattctttgcccagagttgcagttgtaactgttgccaggacacatggccataaataatagaaagaaagctacaaccacaggctgtttgaaagcttcacctcacctttctgcaaggcagaaaaagtatgaaaaaaaccaaggcttttttcagtagcgtcctatggatgtcacattgtacatcaaacaaccttgtgattataaaacgatcctgggaaggagcccctaactagggcaagtcagaaatagccaggctcgcagcagcgcagcgctgtgtctgctgtgtcctggggcctcccttgttccgacctgtcaattctgctgcctgtcacgcgggtggttctgcccatcgcggctgcgggtcaagcatcttcaagggaaggacggactggaggcctcaccgtggactcaactctgcattctccgtgccacattcctccagttcccacacgtagaagggaacgaaactgacgtctacctcatggggctgctgtgtgggtttgggaggcaaaaatctatgaagggttttttgaaatcccataggtgccacatctatgagatgtttgataaatgtgaatatgcttttacatttgggcttatctaatttgcaataagagagcctctctctatcaacaccagcttctctctcgggctgtttgctcagggaaggcaagaaagccacgtgctggccctctgccttctctaaagtgctgttggagcatggaggagctggaggagatggggatggactgacagctaagagggcggctgctgggactagatagtggatgaagaaagaaggacgaggaagccgtggggcagcctcttcacatggggacaggggatggagcatgaggcaagggaaggaaaagcagagcttatttttcacctaaggtggagaaggatcactttacaggcaacgctcattttaagcaacccttaagaaatgtttatgtttctttattaccaatgtaatctatgattattgaaggaaatttagaaaatgcgtagatacaaaattaaaaaaaaatactgtccaaaaa

SEQ ID NO: 2 - protein, 451 aa, Homo sapiens
MLRFYLFISLLCLSRSDAEETCPSFTRLSFHSAVVGTGLNVRLMLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITGLDPAGPLFNGKPHQDRLDPSDAQFVDVIHSDTDALGYKEPLGNIDFYPNGGLDQPGCPKTILGGFQYFKCDHQRSVYLYLSSLRESCTITAYPCDSYQDYRNGKCVSCGTSQKESCPLLGYYADNWKDHLRGKDPPMTKAFFDTAEESPFCMYHYFVDIITWNKNVRRGDITIKLRDKAGNTTESKINHEPTTFQKYHQVSLLARFNQDLDKVAAISLMFSTGSLIGPRYKLRILRMKLRSLAHPERPQLCRYDLVLMENVETVFQPILCPELQL

SEQ ID NO: 3 - DNA, 21 bp, Artificial
  primer
tctgggtttgttggagagatg

SEQ ID NO: 4 - DNA, 20 bp, Artificial
  primer
tcagtgtcggaatggatgac

(19) United States
(12) Patent Application Publication
(10) Pub. No.: US 2016/0369349 A1
(43) Pub. Date: Dec. 22, 2016

(54) NOVEL LUNG CANCER BIOMARKER (LIPH)

(71) Applicants: EIKEN KAGAKU KABUSHIKI KAISHA, Taito-ku, Tokyo (JP); NATIONAL INSTITUTE OF ADVANCED INDUSTRIAL SCIENCE AND TECHNOLOGY, Chiyoda-ku, Tokyo (JP)

(72) Inventors: Yasuhiro SEKI, Shimotsuga-gun, Tochigi (JP); Norio SHIBATA, Shimotsuga-gun, Tochigi (JP); Makoto ASASHIMA, Tsukuba-shi, Ibaraki (JP); Akira KURISAKI, Tsukuba-shi, Ibaraki (JP)

(21) Appl. No.: 14/901,578

(22) PCT Filed: Mar. 18, 2014
(86) PCT No.: PCT/JP2014/058147

§ 371 Date: Dec. 28, 2015

(30) Foreign Application Priority Data
Jun. 28, 2013 (JP) 2013-136820

(51) Int. Cl. C12Q 1/68; G01N 33/574

(52) U.S. Cl. CPC C12Q 1/6886; G01N 33/57488; C12Y 301/01; G01N 33/57423; G01N 33/57407; G01N 2333/918; C12Q 2600/00

(57) ABSTRACT

[Problem to be Solved] To provide a new lung-cancer biomarker, which has a high detection efficiency and determines the type (histological type) of lung cancer.

[Solution] LIPH (lipase, member H), which is enhanced in a plurality of lung cancer-derived cell lines compared to that in normal lung-derived epithelial cells and is almost surely expressed highly in lung cancer.

### TECHNICAL FIELD

The invention of the present application relates to the technical field of laboratory test reagents for assisting the diagnosis of cancer. The invention of the present application further relates to a cancer biomarker, particularly a lung-cancer biomarker for detecting lung cancer and an esophageal cancer biomarker for detecting esophageal cancer.

### BACKGROUND ART

Cancer has increased due to the aging of the population and the like, and it is said that, in 2012 in the world, the number of cancer patients reached 32 million and 14.2 million people per year were newly diagnosed as having cancer. In Japan, cancer is also a disease ranked first as the cause of death, of which 340,000 people per year die. Cancer has become a disease which can be cured if it is detected in early stage, so making the early detection of cancer is important. The development of endoscopy, PET, CT, and the like has been performed for the early detection of cancer; however, the development of cancer biomarkers has become indispensable for efficiently testing a large number of subjects.

Although various cancer biomarkers have been developed, there is a need for the development of additional cancer biomarkers for lung cancer and esophageal cancer.

Lung cancer is one of poor-prognosis diseases which is difficult to detect early and has a 5-year survival rate of less than 20%. However, as for stage IA and IB lung cancer, the 5-year survival rate considerably improves to on the order of 70%. The type (histological type) of lung cancer is pathologically classified into small-cell lung carcinoma and non-small cell lung carcinoma based on its morphology, and the latter is further classified into squamous cell carcinoma, adenocarcinoma, and large cell carcinoma. Biomarkers currently used in common include CYFRA21-1, CEA, SLX, SCC, ProGRP, and NSE. CEA is the most common tumor biomarker and SLX is highly specific to adenocarcinoma. CYFRA21-1 and SCC are considered to be highly specific to squamous cell carcinoma, and ProGRP and NSE are said to be highly specific to small cell carcinoma.

Also, for example, Patent Literature 1 reports that the use of NNMT in combination with any of conventional biomarkers, such as CYFRA21-1, CEA, NSE, and SCC improves sensitivity. Non Patent Literature 1 reports a lung-cancer biomarker targeting a sugar chain.

Esophageal cancer is one of the most mortal malignant tumors in the digestive organs, and its pathology has often already made substantial progress when the esophageal cancer is detected by testing. It is the present situation that even curative treatment at stage 1 still results in a 5-year survival rate as low as 40 to 60%. However, unfortunately, there are at present few esophageal cancer biomarkers useful for even any of diagnosis, therapy evaluation, and prognosis evaluation. SCC and CEA are used for squamous cell carcinoma and CEA is used for adenocarcinoma, and in addition, there are p53 antibody and CYFRA21-1 as esophageal cancer biomarkers.

In addition, Opa interacting protein 5 is reported as a biomarker for esophageal cancer (Non Patent Literature 2).

### CITATION LIST

### Patent Literature

Patent Literature 1: JP Patent Publication (Kohyo) No. 2009-545731

### Non Patent Literature

Non Patent Literature 1: Experimental Medicine, Vol. 25, No. 17 (Extra issue) “Haigan-ni-okeru tosa-hyoteki-shuyo-mahkah-no tansaku (Search for Sugar Chain-Targeting Tumor Biomarker)” Koji Ueda, Yataro Daigo, and Yusuke N akamura.

Non Patent Literature 2: Cancer Sci. 103: 577-586, 2012

### SUMMARY OF THE INVENTION

Clinically used cancer biomarkers, such as CEA, remain to have a low detection rate and, for example, have an early-stage lung cancer detection rate of on the order of 30%; thus, there is a need for more highly sensitive new biomarkers. Accordingly, a first object of the invention of the present application is to provide a new cancer biomarker. A second object of the invention of the present application is to provide a cancer biomarker having high detection efficiency. A third object of the invention of the present application is to provide a new lung-cancer biomarker. A fourth object of the invention of the present application is to provide a lung-cancer biomarker having high detection efficiency. A fifth object of the invention of the present application is to provide a lung-cancer biomarker capable of determining the type (histological type) of lung cancer. Further, a sixth object of the invention of the present application is to provide a lung-cancer biomarker capable of having improved detection efficiency and/or improved detection specificity by combination with other lung-cancer biomarkers.

In addition, a seventh object of the invention of the present application is to provide a new esophageal cancer biomarker. An eighth object of the invention of the present application is to provide an esophageal cancer biomarker having high detection efficiency. Further, a ninth object of the invention of the present application is to provide an esophageal cancer biomarker capable of having improved detection efficiency and/or improved detection specificity by combination with another esophageal cancer biomarker.

The inventors of the present application have found that LIPH (lipase, member H) is enhanced in a plurality of lung adenocarcinoma-derived cell lines compared to that in normal lung-derived epithelial cells and is almost surely expressed highly in lung cancer and esophageal cancer, thereby accomplishing the invention of the present application.

In addition, the inventors of the present application have demonstrated that as a result of immunohistochemistry using a lung cancer tissue microarray, LIPH has shown staining in lung tumor tissues but has not shown staining in interstitial tissues and has also been enhanced protein expression in clinical lung tumor tissue samples. When looked at by histological type of lung cancer, the LIPH protein expression has been positive at rates as high as 70% for lung adenocarcinomas and 70% for bronchiolo-alveolar carcinomas, while the expression has been observed at 30% for lung squamous cell carcinomas, at as slightly low as 20% for large cell carcinomas, and only 1 in 10 specimens for small cell carcinomas; thus, it has been found that the LIPH protein is a protein specifically expressed in lung adenocarcinoma. Based on these findings, the inventors of the present application provide a biomarker for determining the histological type of lung cancer.

The invention of the present application is excellent in that it provides a highly reliable lung-cancer biomarker. In particular, LIPH is an excellent biomarker in that it can specifically detect lung adenocarcinoma.

The inventors of the present application have also carried out immunohistochemistry using an esophageal cancer tissue microarray; as a result, LIPH has been found to be stained in esophageal cancer tissues but not stained in interstitial tissues.

The present specification incorporates the contents of the specification and/or drawings of Japanese Patent Application No. 2013-136820 on which the priority of the present application is based.

### BRIEF DESCRIPTION OF DRAWINGS

FIG. 1 is a graph showing the expression of a lung-cancer biomarker in lung cancer cell lines.

FIG. 2 is a photograph showing the expression of a lung-cancer biomarker, LIPH, in lung cancer tissue.

FIG. 3 is a series of photographs showing the results of observing the expression of LIPH by lung-cancer histological type.

FIG. 4 is a graph showing the detection amount of serum LIPH in healthy volunteers and cancer patients.

FIG. 5 is a graph showing correlation plots with CEA.

FIG. 6 is a photograph showing the results of observing the expression of LIPH by esophageal cancer histological type.

### DETAILED DESCRIPTION

### 1. Preface

To develop a cancer biomarker, the inventors of the present application have first selected candidate genes predicted to be significantly highly expressed in lung cancer. From the candidate genes, a gene which has a high expression level in a plurality of lung cancer-derived cell lines compared to normal lung-derived epithelial cells has further been selected, thereby accomplishing a cancer biomarker.

### 2. Cancer Biomarker Gene, Lung-Cancer Biomarker Gene, and Esophageal Cancer Biomarker Gene

The inventors of the present application have identified LIPH (lipase, member H) gene as a cancer biomarker.

In addition, the inventors of the present application have identified LIPH (lipase, member H) gene as a lung-cancer biomarker and as an esophageal cancer biomarker.

As shown in Examples below, the above gene has been confirmed to be enhanced in a plurality of types of lung cancer-derived cell lines compared to that in normal lung-derived cells. In addition, immunostaining has detected LIPH as a product of the above gene from lung cancer tissues and esophageal cancer tissues. This biomarker is also a biomarker which has a high correlation with prognosis after resection of lung cancer as well as specificity to lung cancer and esophageal cancer and a very high possibility of being used for the prognosis prediction of lung cancer.

3. Confirmation of Expression of Cancer Biomarker Gene, Lung-Cancer Biomarker Gene, and/or Esophageal Cancer Biomarker Gene

Well-known methods for detecting mRNA of a gene or a protein encoded by the gene can be used for the confirmation of the expression of the gene. Methods for confirming the presence of mRNA which can be used include, for example, a LAMP method, a PCR method, and a microarray method. Methods for confirming the protein which can be used include, for example, methods using antibodies, such as an immunostaining method and an ELISA method.

4. Methods for Confirming Presence of Cancer Cell and Assisting Cancer Diagnosis Using Cancer Biomarker, Methods for Confirming Presence of Lung Cancer Cell and Assisting Lung Cancer Diagnosis Using Lung-Cancer Biomarker, and Methods for Confirming Presence of Esophageal cancer Cell and Assisting Esophageal cancer Diagnosis Using Esophageal cancer Biomarker

4-1. Sample

Any sample can be used as a sample for use in the methods for confirming the presence of cancer cells, lung cancer cells, or esophageal cancer cells; however, preferred examples thereof can include samples collected from subjects and specific examples thereof can include any collected body fluids and any biopsy samples. Particularly preferred examples can include biopsy samples of affected areas of the lung or affected areas of the esophagus or sputum samples from subjects.

4-2. Method for Detecting mRNA in Sample and Reagent and Kit Therefor

For example, cells in each biopsy sample can be lysed using a surfactant, a homogenizer, or a conventional method such as freeze-thawing to extract the total RNA. The quantification of desired mRNA can be used a LAMP method, a differential display method, a method using a DNA array (DNA chip), a quantitative PCR method, a real-time PCR method, or a competitive PCR method.

Examples of the real-time PCR method include a method which involves monitoring the formation of the amplified product of the biomarker gene in real-time for analysis. Examples of the real-time monitoring reagent include SYBRGreen I and TaqMan probe.

Examples of the competitive PCR method include a method which involves synthesizing cDNA from the total RNA or mRNA in cells using a reverse transcriptase and to react the cDNA with a DNA competitor in the same tube and a method which involves further adding an RNA competitor together with mRNA during the reverse transcription for reaction. The internal sequence other than the primer sequences of the competitor may be, for example, a sequence homologous or non-homologous to the sequence of mRNA to be amplified.

When the use of the real-time PCR method is specifically exemplified, cDNA can be synthesized from the extracted total RNA by reverse transcription using oligo-dT primers and measured by real-time PCR. For example, ISOGEN method (Nippon Gene Co., Ltd.) can be used for RNA extraction. For example, a PrimeScript(R) II 1st strand cDNA Synthesis Kit can be used for reverse transcription. For example, THUNDERBIRD (R) SYBR Mix (Toyobo Co., Ltd.) or TaqMan probe can be used for real-time PCR to measure the amplified product.

A hybridization method is also included which uses a labeled probe having a sequence complementary to the sequence of the LIPH gene or to a sequence in which one to several nucleotides are deleted, substituted, and/or added in the sequence.

The primers may be any primer set provided that it is a primer set enabling the amplification of the LIPH gene as a cancer biomarker gene, a lung-cancer biomarker gene, and/or an esophageal cancer biomarker gene by a gene amplification method, such as a PCR method. A primer can be used which consists of any consecutive 15 or more nucleotides, preferably consecutive 16 to 30 nucleotides, more preferably consecutive 18 to 28 nucleotides in the LIPH gene, for example, the sequence represented by SEQ ID NO: 1 or a sequence in which one or several nucleotides are deleted, substituted, and/or added in the sequence. Specifically, the primers described in Table 1 below can be used.

TABLE 1

| | | |
|---|---|---|
| LIPH | forward | TCTGGGTTTGTTGGAGAGATG |
| | reverse | TCAGTGTCGGAATGGATGAC |

The differential display method or the DNA array (DNA chip) can also be used.

In addition, the hybridization method can use a labeled probe having a sequence complementary to the sequence represented by SEQ ID NO: 1, or to a sequence in which one or several nucleotides are deleted, substituted, and/or added in the sequence.

Examples of the label for the probe can include a radioactive isotope, a fluorescent substance, a luminescent substance, and an enzyme. Examples of the radioactive isotope which can be used include [32P], [33P], [125I], and [131I]. Examples of the fluorescent substance which can be used include Cy2, Cy3, Cy5, Cy5.5, Cy7, fluorescein isothiocyanate, fluorescamine, and rhodamine. Examples of the enzyme include alkaline phosphatase, peroxidase, β-galactosidase, β-glucosidase, and malate dehydrogenase which are commonly used as labels.

4-2-1

Examples of the kit for performing the mRNA detection method include (1) a kit containing primers or a set of primers (primer set) enabling the amplification of LIPH (lipase, member H) lung-cancer or esophageal cancer biomarker gene by a gene amplification method, such as a LAMP method and (2) as an example, a kit containing the set of primers (primer set) given in Table 1.

4-3. Method for Detecting Protein in Sample

According to the invention of the present application, the method for detecting the biomarker protein may be any method provided that it is a method capable of specifically detecting the biomarker protein, such as a detection method using antibody(ies) or a mass spectrometry method. For the method using antibody(ies), the antibody(ies) to the protein encoded by the LIPH gene may be one already marketed or may be prepared by a conventional method. Examples of the method for detecting the biomarker protein using antibody(ies) include an immunostaining method, an immunofluorescent method, various enzyme immunoassays, radioimmunoassay (RIA)/enzyme-linked immunoassay (ELISA) methods, a double monoclonal antibody sandwich immunoassay method, monoclonal polyclonal antibody(ies) sandwich assay method, a western blotting method, a biotin-avidin method, and an immunoprecipitation method.

4-3-1

Examples of the kit for performing the method for detecting a protein described in 4. 3 above include the following.

(1) (i) enzyme-labeled monoclonal antibody(ies) and (ii) a substrate solution

Examples of the kit for performing the sandwich ELISA method include one containing the following reagents.

(2) (i) monoclonal antibody(ies) or polyclonal antibody(ies), (ii) enzyme-labeled monoclonal antibody(ies) or polyclonal antibody(ies), and (iii) a substrate solution

The kit for performing the biotin-avidin method contains the following reagents.

(3) (i) biotinylated monoclonal antibody(ies) or polyclonal antibody(ies), (ii) enzyme-labeled avidin or streptavidin, and (iii) a substrate solution

The kit for performing the sandwich ELISA method and the biotin-avidin method contains the following reagents.

(4) (i) monoclonal antibody(ies) or polyclonal antibody(ies), (ii) biotinylated monoclonal antibody(ies) or polyclonal antibody(ies), (iii) enzyme-labeled avidin or streptavidin, and (iv) a substrate solution

The substrate solution refers to a solution containing a substrate for the enzyme labeled on the antibody(ies), the substrate producing a detectable change by enzyme reaction. Examples of the substrate solution which can be used include a p-nitrophenylphosphate-containing buffer solution in the case of labeling with alkaline phosphatase (AP), an o-phenylenediamine-containing buffer solution in the case of labeling with horseradish peroxidase (HRPO), and a 4-methylumbelliferyl-β-galactoside-containing buffer solution in the case of labeling with β-galactosidase.

The method using biotinylated antibody(ies) may be a well-known method; however, preferred is a method in which conjugations of streptavidin and peroxidase bound to biotin. Examples of the peroxidase include horseradish peroxidase. In addition, the detection of peroxidase preferably uses a substance developing color by the action of the peroxidase.

The invention of the present application will be more specifically described below based on Examples. However, the present application is not intended to be limited to the following Examples.

### EXAMPLE 1

The inventors of the present application obtained 8 new lung-cancer biomarker candidate factors by a preliminary study for first selecting candidate genes predicted to be significantly highly expressed in lung cancer. In order to verify the expression of these factors in lung cancer, lung cancer derived cell lines were used to perform qPCR to verify the mRNA expression of the candidate factors. Using the culture media described in Table 2, the 16 types of lung cancer-derived cell lines and 2 types of normal lung-derived cells described in the same table were cultured to confluence in 6-well plates. Then, RNA extraction was carried out using ISOGEN (Nippon Gene Co., Ltd.) or RNA easy mini kit (QIAGEN Co., Ltd.). With 500 ng of the extracted total RNA as a template, cDNA was synthesized using PrimeScriptII 1st strand synthesis kit (Takara Bio Inc.). With the synthesized cDNA as a template, qPCR was carried out using THUNDERBIRD SYBR Mix (Toyobo Co., Ltd.).

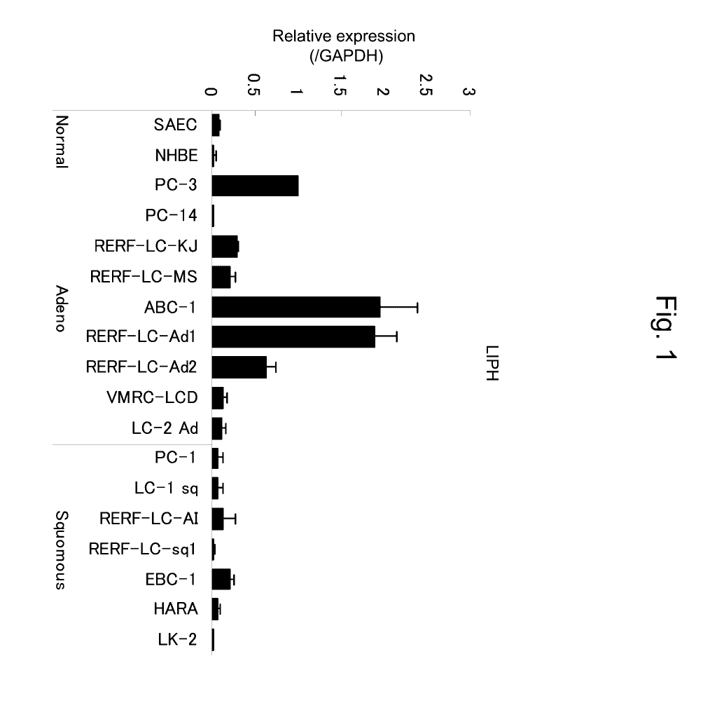

The results for LIPH (lipase, member H) expression are shown in FIG. 1. Two-fold or more expression was observed in a plurality of lung adenocarcinoma cell lines compared to that in normal lung-derived epithelial cells (SAEC: small airway epithelial cells, NHBE: alveolar epithelial cells). Expression increased in lung squamous cancer cell lines. The ordinate of the graph in the figure shows a value provided by dividing the expression level of LIPH gene by the expression level of an internal standard gene, GAPDH, in each cell as a value relative to the expression level in PC-3 cells.

TABLE 2
Cell and Culture Medium Used for Study of Expression of Lung-Cancer
Marker Candidate Factor

| Cell Name | Culture Condition | Origin | Source |
|---|---|---|---|
| Normal Human Small | Small Airway Epithelial Cell | — | Takara |
| Airway Epithelial Cell | Culture Medium Kit (Serum-Free) | | Bio Inc. |
| (SAEC) | (SAGM ™ BulletKit ™) | | (Lonza) |
| Normal Human Bronchial | Bronchial Epithelial Cell | — | Takara |
| Epithelial Cell | Culture Medium Kit (Serum-Free) | | Bio Inc. |
| (NHBE) | (BEGM ™ BulletKit ™) | | (Lonza) |
| PC-3 | EMEM + 10% FCS | Lung Adenocarcinoma | HSRRB |
| PC-14 | RPMI1640 + 10% FCS | Lung Adenocarcinoma | IBL |
| RERF-LC-KJ | RPMI1640 + 10% FCS | Lung Adenocarcinoma | BRC |
| RERF-LC-MS | RPMI1640 + 10% FCS | Lung Adenocarcinoma | HSRRB |
| ABC-1 | EMEM + 10% FCS | Lung Adenocarcinoma | HSRRB |
| RERF-LC-Ad1 | RPMI1640 + 10% FCS | Lung Adenocarcinoma | HSRRB |
| RERF-LC-Ad2 | RPMI1640 + 10% FCS | Lung Adenocarcinoma | HSRRB |
| VMRC-LCD | EMEM + 10% FCS | Lung Adenocarcinoma | HSRRB |
| LC-2 Ad | (HamF12:RPMI1640 = 1:1) + | Lung Adenocarcinoma | BRC |
| | 15% FBS + 25 mM HEPES | | |
| | (pH 7.2) | | |
| PC-1 | RPMI1640 + 10% FCS | Lung Squamous Cell | IBL |
| | | Carcinoma | |
| LC-1 sq | (HamF12:RPMI1640 = 1:1) + | Lung Squamous Cell | BRC |
| | 10% FBS + 25 mM HEPES | Carcinoma | |
| | (pH 7.2) | | |
| RERF-LC-Al | RPMI1640 + 10% FCS | Lung Squamous Cell | BRC |
| | | Carcinoma | |
| RERF-LC-sq1 | RPMI1640 + 10% FCS | Lung Squamous Cell | HSRRB |
| | | Carcinoma | |
| EBC-1 | EMEM + 10% FCS | Lung Squamous Cell | HSRRB |
| | | Carcinoma | |
| HARA | RPMI1640 + 10% FCS | Lung Squamous Cell | HSRRB |
| | | Carcinoma | |
| LK-2 | RPMI1640 + 10% FCS | Lung Squamous Cell | HSRRB |
| | | Carcinoma | |

Abbreviated Name of Source
IBL Immunobiology Laboratory
HSRRB Human Science Research Resources Bank
BRC Riken Bioresource Center

### EXAMPLE 2

To verify the protein expression, an anti-LIPH rabbit polyclonal antibody from Proteintech Co., Ltd. was used to perform immunostaining on a commercial tissue microarray (Outdo Co., Ltd., Shanghai). Formalin-fixed paraffin-embedded sections of 60 specimens of lung-cancer tissue and 60 specimens of adjacent normal tissue derived from the same donors as those of the cancer specimens were spotted on the tissue microarray used. The number of specimens by lung-cancer type was 10 each for lung squamous cell cancer, lung adenocarcinoma, lung adenosquamous carcinoma, bronchiolo-alveolar carcinoma, large cell carcinoma, and small-cell lung carcinoma.

Deparafinization and hydrophilization were first carried out by performing 10-minute xylene treatment twice and 10-minute 100% EtOH (ethanol) treatment twice, then 10-minute 90% EtOH treatment, and finally 10-minute 70% EtOH ethanol treatment.

Antigen retrieval was then carried out by autoclaving in a 10 mM citrate buffer solution at 110° C. for 10 minutes. Treatment with 0.3% H₂O₂/MeOH was performed for 30 minutes to inactivate endogenous peroxidase. Thereafter, Vectastain ABC Kit (Vector Co., Ltd.) was used to perform immunostaining, and DAB Peroxidase Substrate Kit (Vector Co., Ltd.) was used to perform color development. After immunostaining, counterstain was also carried out by using hematoxylin (Sigma Co., Ltd.).

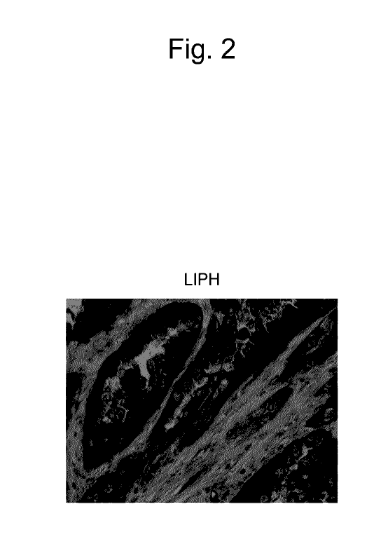

FIG. 2 shows the results of observation and photographs with a microscope (BX50 (Olympus Co., Ltd.)) and EOS kiss X6i. It showed that LIPH was strongly expressed in the cytoplasm of tumor cells in lung adenocarcinoma tissues.

### EXAMPLE 3

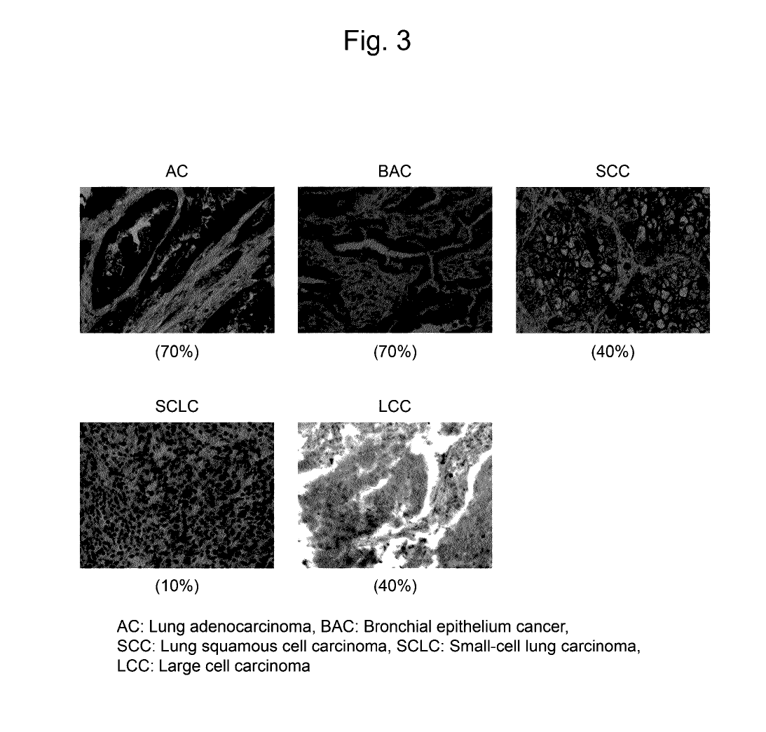

From the results of Examples 1 and 2, it was determined that LIPH was promising as a new lung-cancer biomarker candidate. Accordingly, the percentage of highly expressed LIPH was examined below by lung cancer histological type, and the results are shown (FIG. 3). The numeral values in the figure indicate at what percentage LIPH was highly expressed in 10 specimens spotted on the tissue microarray. As a result, the expression could be observed at a percentage as high as 70% in lung adenocarcinomas, and could also be observed at a percentage as high as 70% in bronchiolo-alveolar carcinomas (BACs) showing a papillary finding similar to that in adenocarcinoma. On the other hand, the expression was observed at 30% in squamous cell carcinomas, at a slightly low of 20% in large cell carcinomas, and in only 1 of 10 specimens in small-cell lung carcinomas, suggesting that it was specific to non-small cell lung carcinoma. For tumor cells, the cytoplasm was positive in lung adenocarcinomas; the cell surface in bronchial epithelium cancers; the membrane and the nucleus in squamous cell carcinomas; and mainly the nucleus in small-cell lung carcinomas.

### EXAMPLE 4

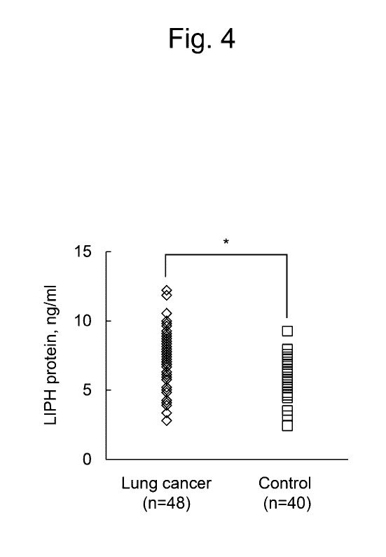

Lung cancer patients' serum were purchased from Bizcom Japan Inc., and a healthy volunteers' serum specimens were procured from within Eiken Chemical Co., Ltd. In Maxisoap (Nunc Co., Ltd.), coated the well with 100 kg/well of an anti-LIPH rabbit polyclonal antibody (Proteintech Co., Ltd.) by incubating at 4° C. overnight, and blocking was carried out at 4° C. overnight using 25% Block Ace. After removing the blocking solution, 100 μg/well of the serum specimen diluted ten-fold in 10% Block Ace was added and incubated at room temperature for 1 hour. After washing 3 times with 10 mM PBS (pH 7.2), an anti-LIPH mouse polyclonal antibody (Sigma Co., Ltd.) diluted 1:500 was added and incubated at room temperature for 1 hour. After washing 3 times, HRP-labeled anti-mouse IgG was added and incubated for 1 hour, followed by adding 100 μl of OPD substrate to develop Absorbance at 492 nm was read to quantify LIPH. As a standard, the standard substance included in Human Lipase member H ELISA Kit from Cusabio Biotech Co., Ltd was used. The results are shown in FIG. 4.

### EXAMPLE 5

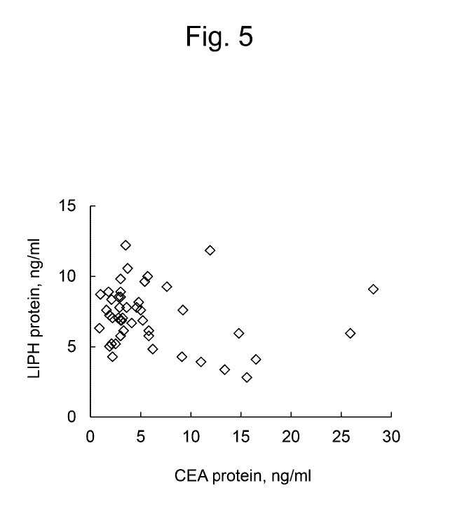

The CEA values of the serum specimens used in Example 4 were measured. E Test “TOSOH” II CEA immunoreactive reagent (Tosoh Corporation) and AIA-800 (Tosoh Corporation) were used for determining CEA values. A scatter diagram was drawn, in which the serum LIPH values measured in Example 4 were plotted on the X-axis and serum CEA values were plotted on the Y-axis, and a correlation coefficient was determined. The results are shown in FIG. 5.

### EXAMPLE 6

To verify the protein expression of LIPH in esophageal cancer tissue, an anti-LIPH rabbit polyclonal antibody from Proteintech Co., Ltd. was used to perform immunostaining on a commercial tissue microarray (Outdo Co., Ltd., Shanghai). Formalin-fixed paraffin-embedded sections of 6 specimens of esophageal cancer tissue and 6 specimens of adjacent normal tissue derived from the same donors as those of the cancer specimens were spotted on the tissue microarray used. The histological types of esophageal cancer specimens were all squamous cell cancer.

Deparafinization and hydrophilization were first carried out by performing 10-minute xylene treatment twice and 10-minute 100% EtOH (ethanol) treatment twice, then 10-minute 90% EtOH treatment, and finally 10-minute 70% EtOH ethanol treatment.

Antigen retrieval was carried out by heating treatment at 100° C. for 20 minutes in a 10 mM citrate buffer solution (pH 6.0), and slide treatment was carried out for 30 minutes using 0.3% hydrogen peroxide/methanol. Staining used a biotin-free immunostaining intensifying system utilizing tyramide, CASH (Dako Co., Ltd.) and was carried out according to the manufacturer's protocol. Color development was carried out using DAB Peroxidase Substrate Kit (Vector Co., Ltd.), followed by counterstain with hematoxylin (Sigma Co., Ltd.). Observation was performed using a microscope, IX59 (Olympus Co., Ltd.), and images were obtained using EOS Kiss X6i (Canon Co., Ltd.).

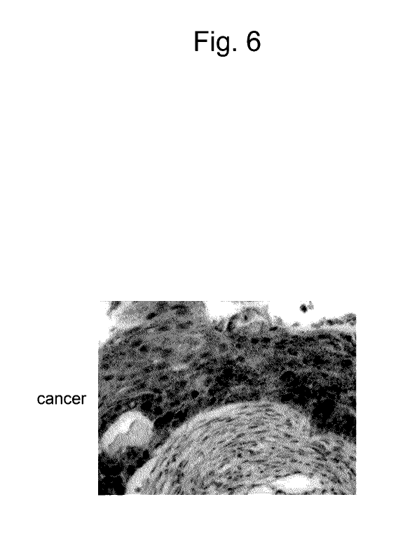

The results are shown in FIG. 6. FIG. 6 demonstrated that LIPH could also be used as an esophageal cancer biomarker.

### INDUSTRIAL APPLICABILITY

The invention of the present application can be used in industry producing diagnostic reagents and diagnostic kits. More specifically, the invention can be used in industries producing tumor diagnostic reagents and diagnostic kits.

All publications, patents, and patent applications cited in this specification are intended to be incorporated herein by reference in their entirety.

1-15. (canceled)

16. A method for detecting a cancer comprising:
(i) obtaining a sample from a subject and a healthy volunteer;
(ii) determining at least one of the following cancer markers:
(a) an amount of a polypeptide encoded by LIPH gene in the sample from the subject and an amount of the polypeptide in the healthy volunteer,
(b) an amount of mRNA encoding LIPH in the sample from the subject and an amount of the mRNA encoding LIPH in the healthy volunteer, and
(c) an amount of a polypeptide encoded by LIPH gene in the sample from the subject and the healthy volunteer; and
(iii) comparing:
(1) the amount of the cancer marker in the sample from the subject and amount of the cancer marker in the healthy volunteer as determined in (a) or (b), or
(2) the amount of the polypeptide encoded by LIPH gene in the sample from the subject and the healthy volunteer as determined in (c).

17. The method of claim 16, wherein the cancer is lung cancer or esophageal cancer.

18. A labeled cancer detection probe having a sequence complementary to the sequence represented by SEQ ID NO: I or to a sequence in which one to several nucleotides are deleted, substituted, and/or added in the sequence.

19. The cancer detection probe of claim 18, wherein the cancer is lung cancer or esophageal cancer.

20. A method for detecting a cancer comprising:
(i) obtaining a sample from a subject and a healthy volunteer;
(ii) preparing:
(a) cDNA from mRNA of LIPH gene in the sample from the subject and the healthy volunteer using at least a primer comprising consecutive 15 or more nucleotides in the sequence of LIPH gene, or
(b) cDNA from mRNA of LIPH gene in the sample from the subject and the healthy volunteer using at least a primer comprising any consecutive 15 or more nucleotides in the sequence represented by SEQ ID NO: I or a sequence in which one or several nucleotides are deleted, substituted, and/or added in the sequence;
(iii) determining an amount of cDNA of the LIPH gene in the sample from the subject and the healthy volunteer; and
(iv) comparing the amount of the cDNA of LIPH gene in the sample from the subject and the healthy volunteer.

21. The method of claim 20, wherein the cancer is lung cancer or esophageal cancer.

22. A method for detecting a cancer comprising:
(i) obtaining a sample from a subject and a healthy volunteer,
(ii) contacting an antibody(ies) to LIPH with the sample from the subject and the healthy volunteer.

23. The method of claim 22, wherein the cancer is lung cancer or esophageal cancer.

24. The method of claim 22, wherein the antibody(ies) is a monoclonal antibody.

25. The method of claim 23, wherein the antibody is a monoclonal antibody(ies).

26. A lung cancer detection kit comprising:
(i) a primer comprising consecutive 15 or more nucleotides in the sequence of LIPH;
(ii) a primer comprising any consecutive 15 or more nucleotides in the sequence represented by SEQ ID NO: I or a sequence in which one or several nucleotides are deleted, substituted, and/or added in the sequence; or
(iii) an antibody(ies) to LIPH, or monoclonal antibody(ies) to LIPH.
